(19) United States
(12) Patent Application Publication
(10) Pub. No.: US 2006/0204950 A1
(43) Pub. Date: Sep. 14, 2006

(54) Biological sample preservation, transportation and storage device

(76) Inventors: Ty Ilercil, Mesa, AZ (US); Alp Ilercil, Mesa, AZ (US)

Correspondence Address: GARY L. EASTMAN, EASTMAN & ASSOCIATES, 707 BROADWAY STREET, SUITE 1800, SAN DIEGO, CA, 92101, US

(21) Appl. No.: 11/364,875

(22) Filed: Feb. 27, 2006

Related U.S. Application Data
(60) Provisional application No. 60/656,643, filed on Feb. 25, 2005.

(51) Int. Cl. A01N 1/02; C12M 1/34

(52) U.S. Cl. 435/1.1; 435/284.1; 435/287.2

(57) ABSTRACT

The biological transport and storage device includes a cooling chamber containing one or more biological samples in a sample holder surrounded by coolant air which is intermittently circulated through the cooling chamber across a thermoelectric unit that can cool or heat the circulating air. A fan is used to circulate air through the cooling chamber, and a separate fan may be used to introduce and circulate ambient air over the thermoelectric unit in incoming air chamber. A sample rotator motor may be used to rotate the samples within the cooling chamber. A control system includes a PLC controller that receives temperature inputs from the sample holder, ambient air within the cooling chamber, and the thermoelectric unit, as well as possibly within the sample. The PLC receives these temperature inputs, and controls the fans, sample rotator motor, and thermoelectric units such that the temperature of the samples within the cooling chamber are cooled at a predetermined and selectable temperature profile. This profile may be selected through the PLC interface based upon a number of factors, such as the type of sample (equine semen, bovine semen, embryo, cardiac tissues, etc.) the expected period for transport or delay until use, for example.

### RELATED APPLICATIONS

This application claims the benefit of priority to U.S. Provisional Patent Application Ser. No. 60/656,643 filed Feb. 25, 2005, entitled “Biological Sample Transport and Storage Device” and currently co-pending.

### FIELD OF THE INVENTION

This invention relates generally to devices for the storage of viable living tissue. This invention is more particularly, though not exclusively, useful as a biological sample storage and transport device wherein a biological sample can be successfully stored in a controlled chilled environment and returned to normal temperature for use.

### BACKGROUND OF THE INVENTION

It is often necessary to harvest, preserve, and transport biological samples. One such application is in the field of equine artificial insemination in which semen must be collected from a stallion, preserved by cooling to an appropriate temperature, and transported in the cooled state to another location for insertion into a mare. Over the years, several devices have been used to cool and transport the collected semen, however, these devices typically use liquid nitrogen as the cooling agent, and do not precisely control the temperature of the collected semen. Specifically, these devices cannot sustain the semen at a constant temperature for an extended period of time. Currently available transport devices are available under the Bio-Flite, Equitainer, and ExpectaFoal trade names.

### SUMMARY OF THE INVENTION

The present invention includes a cooling and transportation device having a cooling chamber containing one or more biological samples, such as semen, in a sample holder. These samples are surrounded by coolant air which is intermittently circulated through the cooling chamber across a thermoelectric unit that can cool or heat the circulating air. A fan is used to circulate air through the cooling chamber, and a separate fan may be used to introduce and circulate ambient air over the thermoelectric unit. A sample rotator motor may be used to rotate the samples within the cooling chamber.

A control system includes a PLC controller that receives temperature inputs from the sample holder, ambient air within the cooling chamber, and the thermoelectric unit, as well as possibly within the sample. The PLC receives these temperature inputs, and controls the fans, sample rotator motor, and thermoelectric units such that the temperature of the samples within the cooling chamber are cooled at a predetermined and selectable temperature profile. This profile may be selected through the PLC interface based upon a number of factors, such as the type of sample (equine semen, bovine semen, embryo, cardiac tissues, etc.) the expected period for transport or delay until use, for example. The PLC, through the selective control of the fans, motor, and thermoelectric unit, can control the temperature of the biological sample to within a small fraction of a degree, and may adjust the temperature of the sample as a function of time, regardless of outside, or ambient, temperatures.

In light of the above, it is advantageous to provide a device which has the ability to precisely select and achieve the temperature of a biological sample; the ability to program the device with a predetermined temperature profile which will follow that profile to within a fraction of a degree; incorporates “fuzzy logic” to anticipate the temperature responses of the system to most closely follow the desired temperature profile with very small temperature variations; the ability to maintain very low metabolic rates through the precise cooling of a biological sample, thereby increasing the rate of viability of that sample; the ability to control the cooling rate of a biological sample to avoid temperature shock problems with “immersion” type cooling systems; the ability to precisely control the temperature of a biological sample regardless of the outside, or ambient, temperature; a biological sample transport device having an active temperature control function as compared to the currently available steady state cooling devices; a biological sample preservation device that is pre-programmed for samples to ensure increased viability of a particular sample type or types; and a biological sample and transport system that greatly increases the success rates for artificial insemination through the controlled cooling and heating of the biological sample.

### BRIEF DESCRIPTION OF THE DRAWING

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a block diagram showing the cooling chamber containing one or more biological samples, equipped with a thermoelectric cooling or heating device and an array of temperature sensors; interconnected with a programmable logic controller (PLC) used to precisely control the temperature within the cooling chamber to follow a predetermined cooling profile corresponding to the biological sample within the chamber; and

FIGS. 2-34 are photographic representations of the biological sample transport and storage device of the present invention showing the various components and their interconnectivity.

### DETAILED DESCRIPTION OF A PREFERRED EMBODIMENT

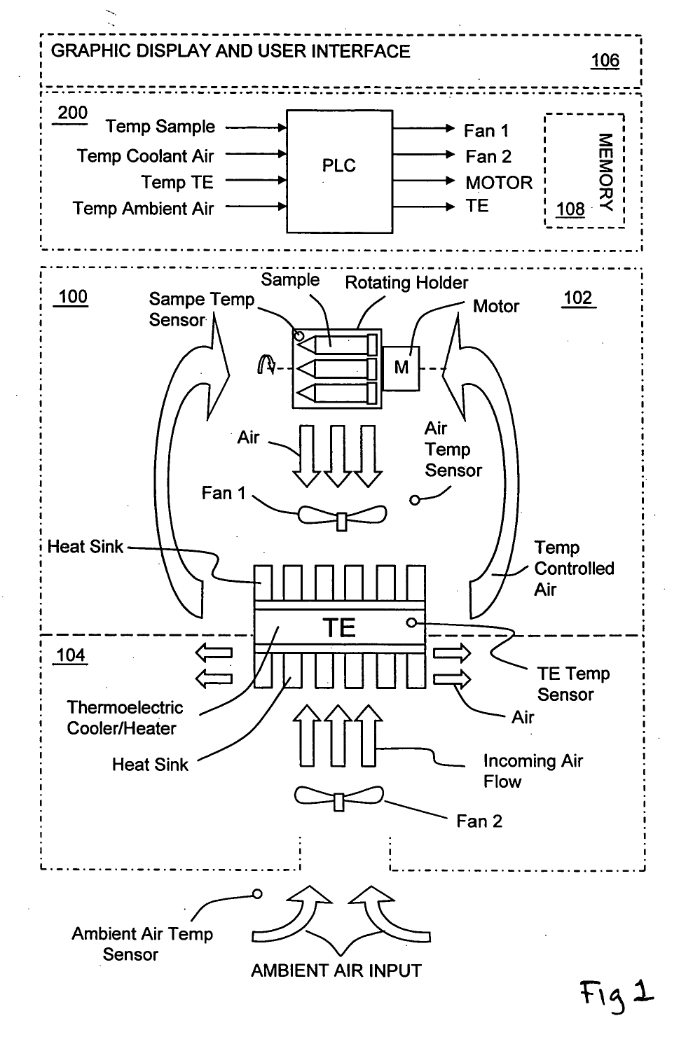

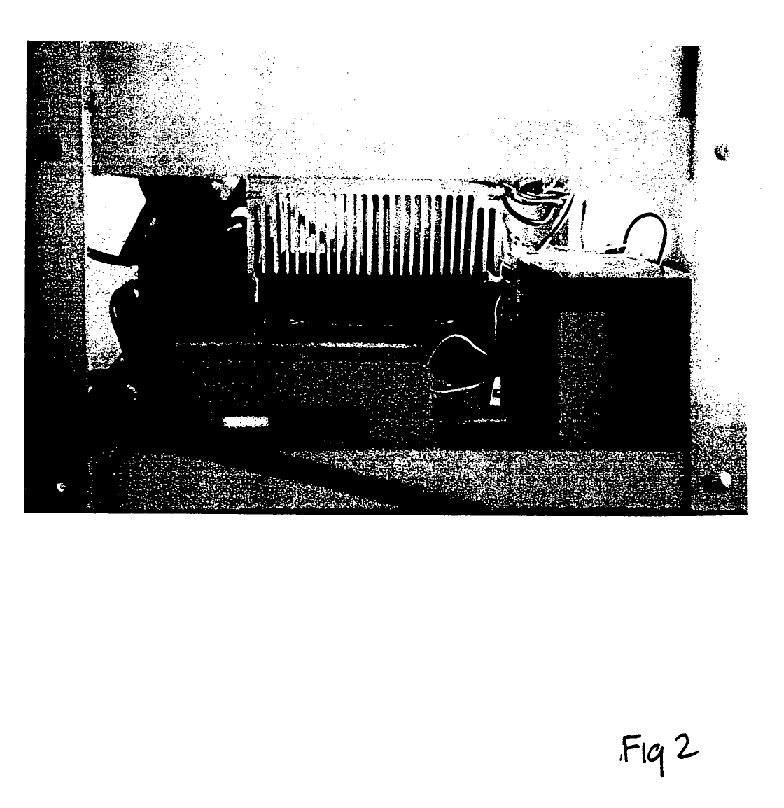

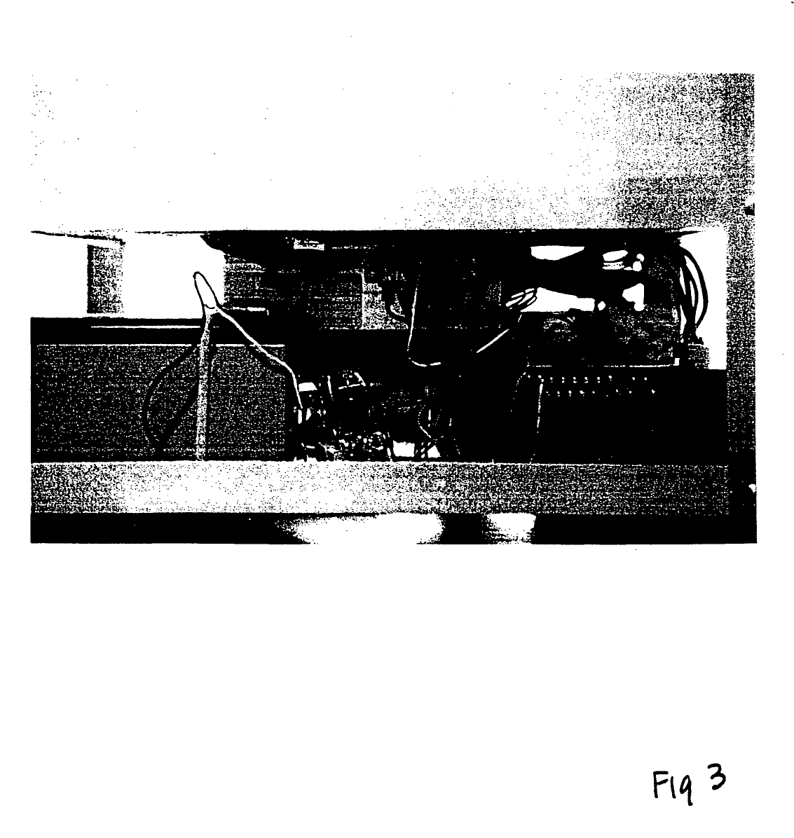

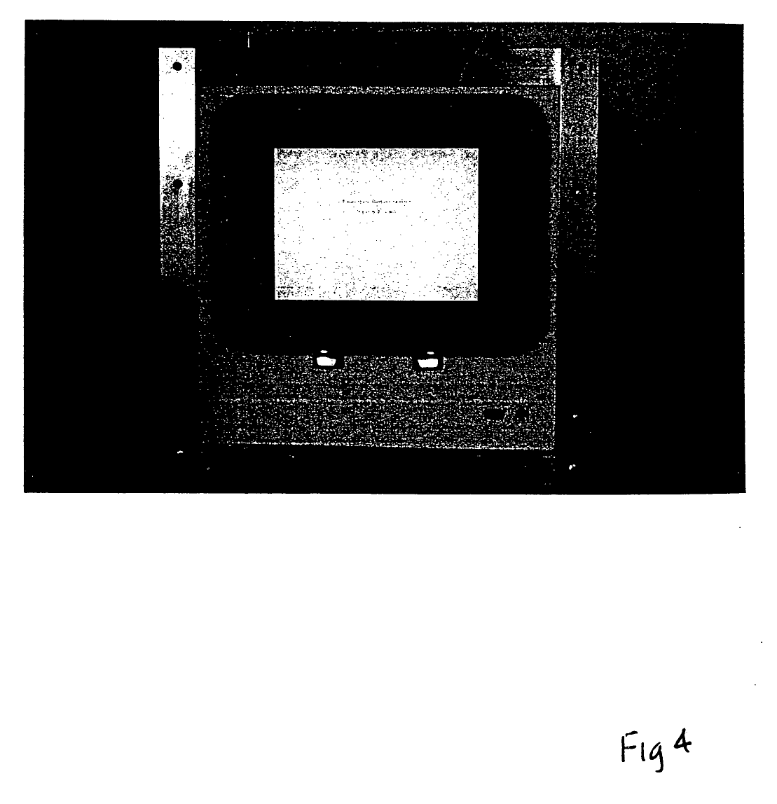

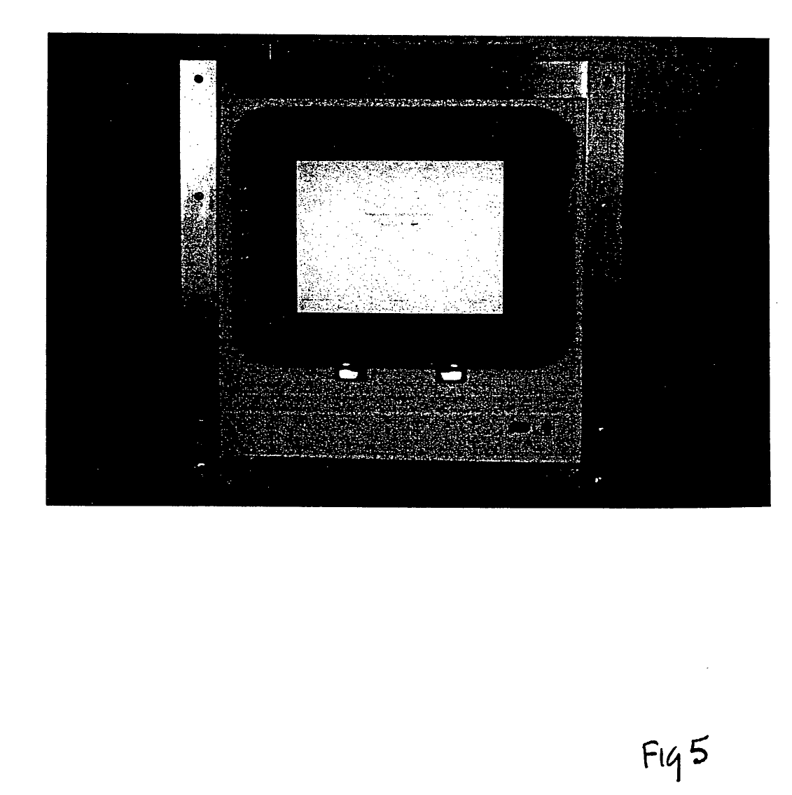

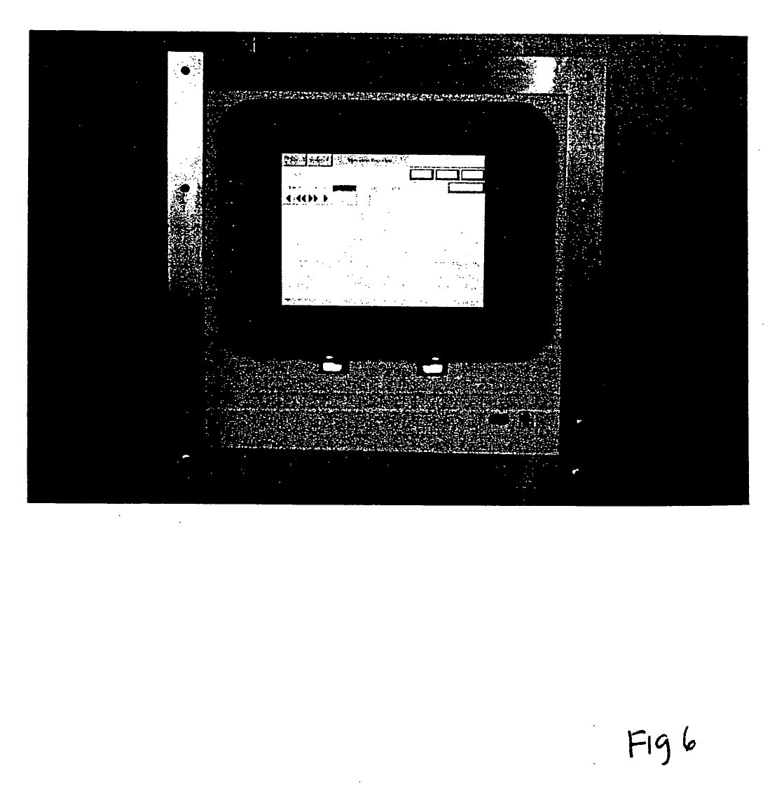

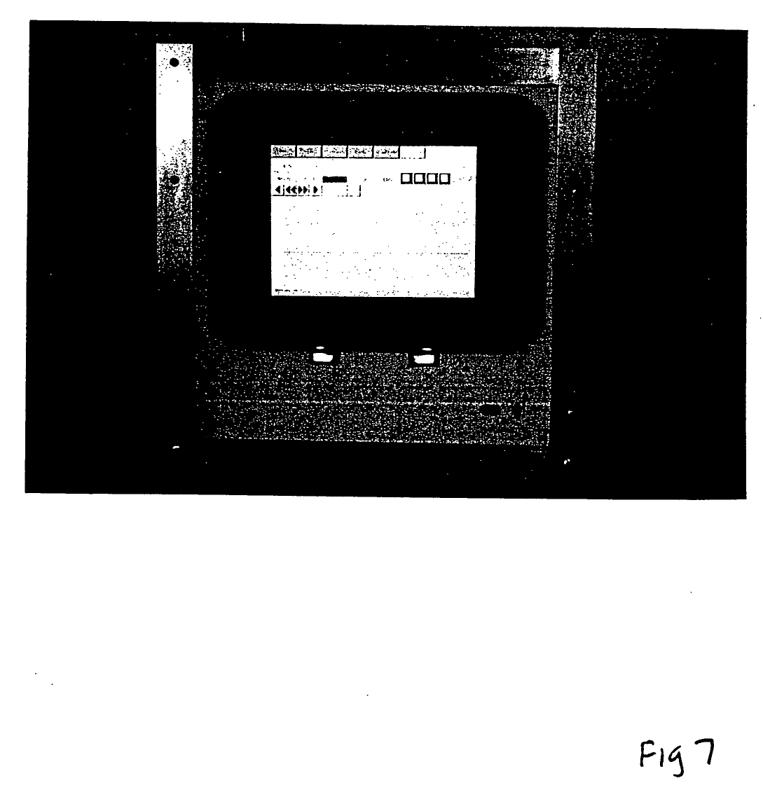

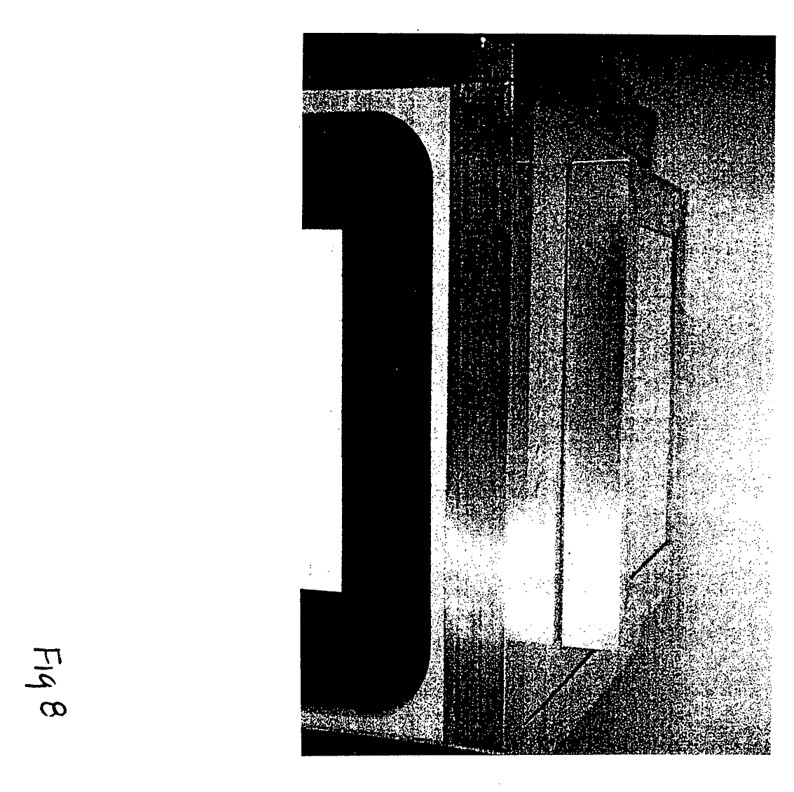

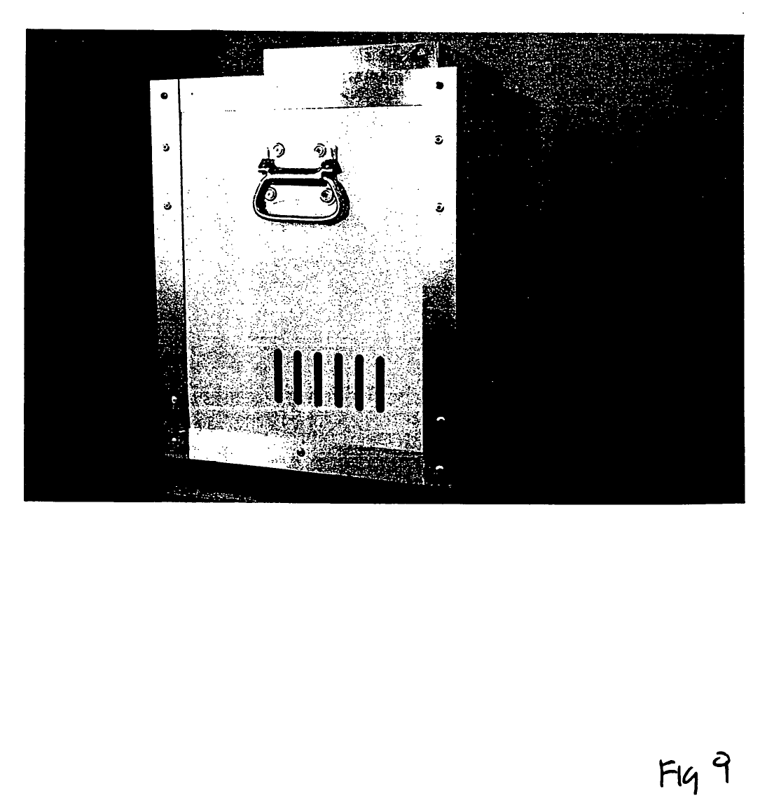

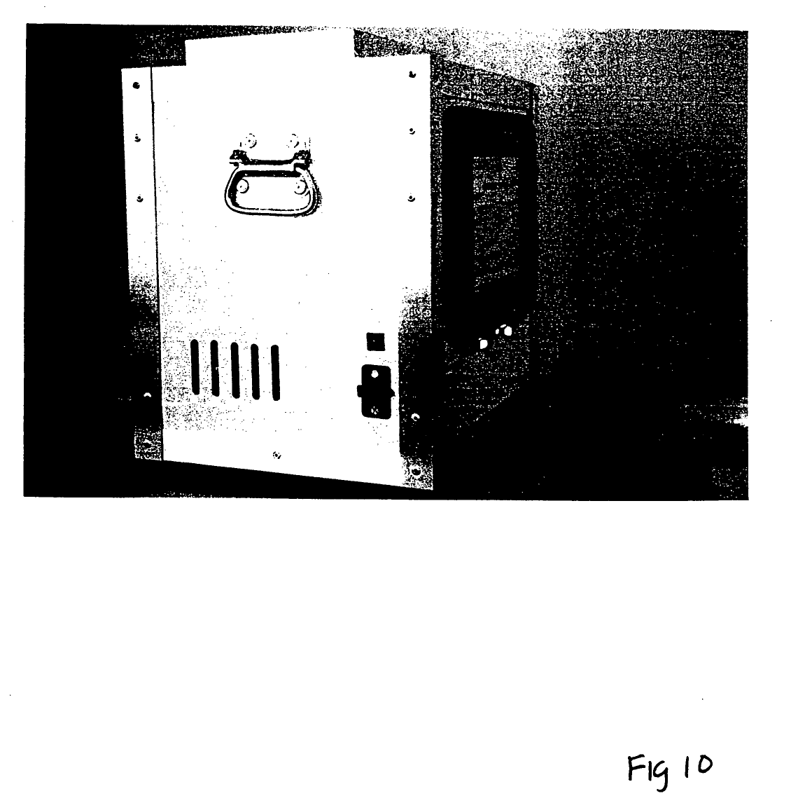

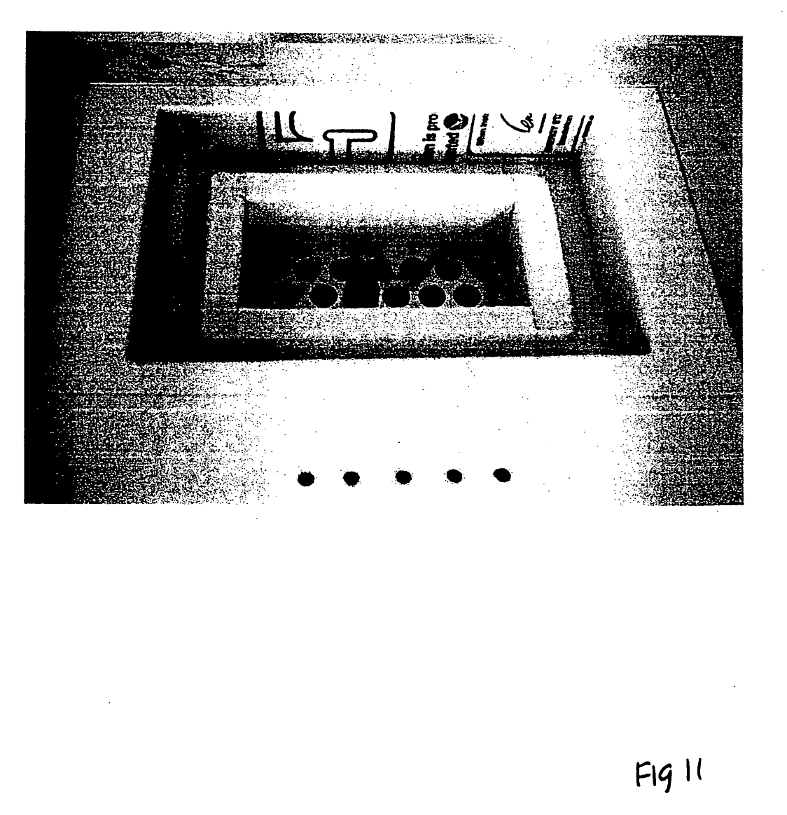

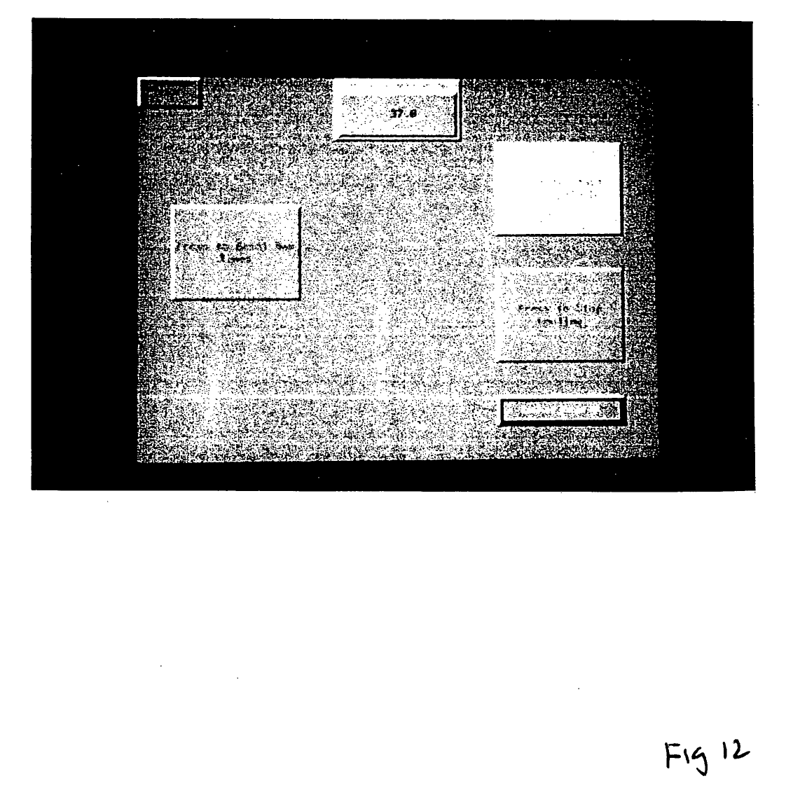

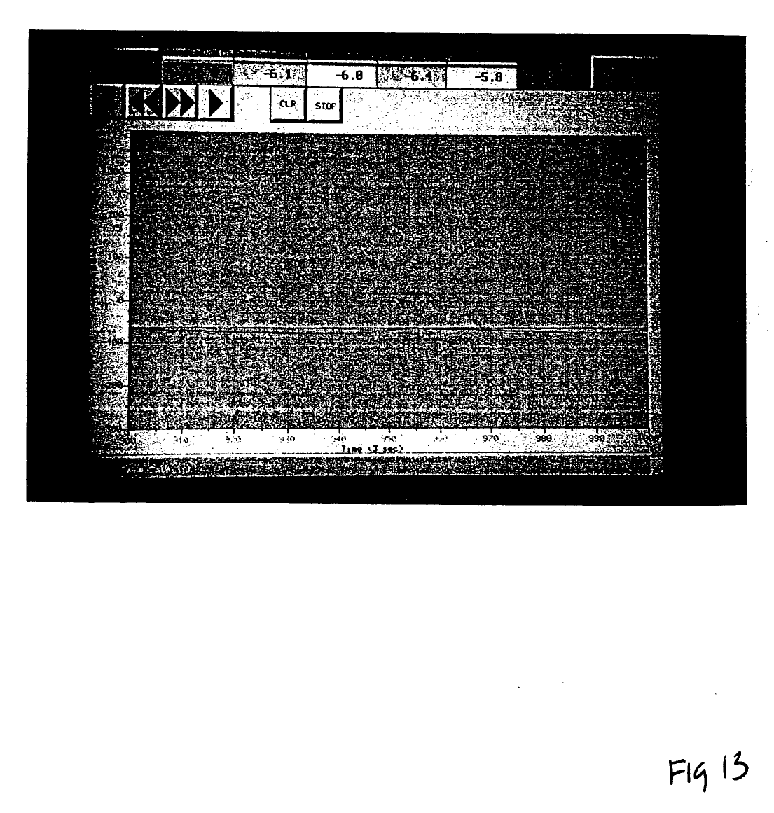

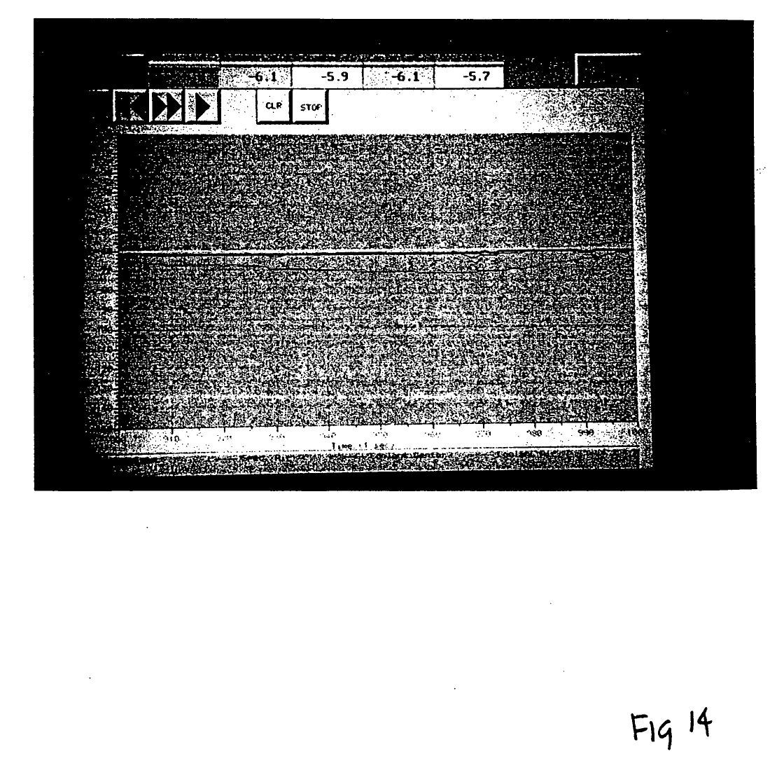

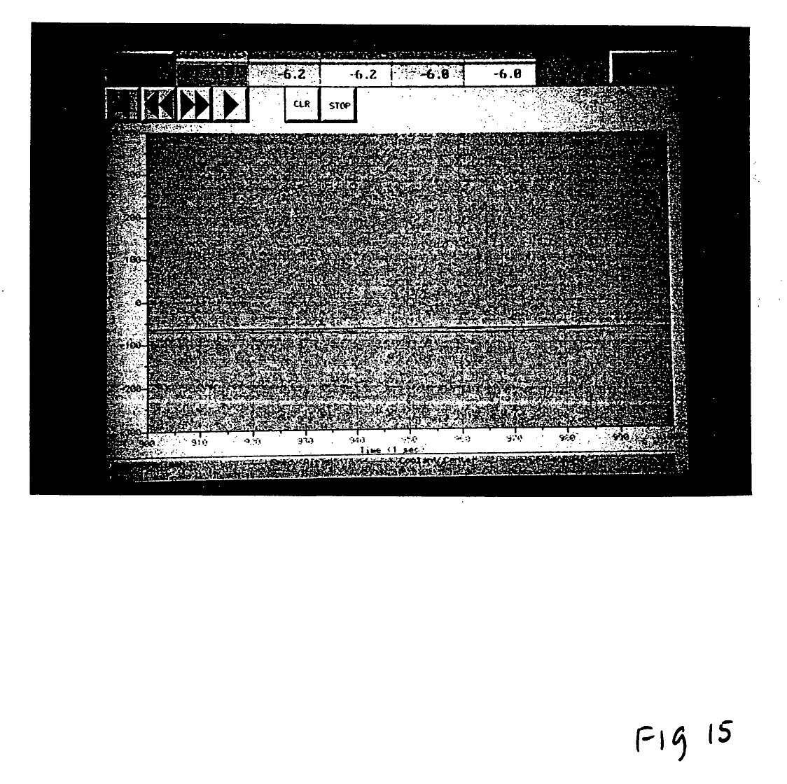

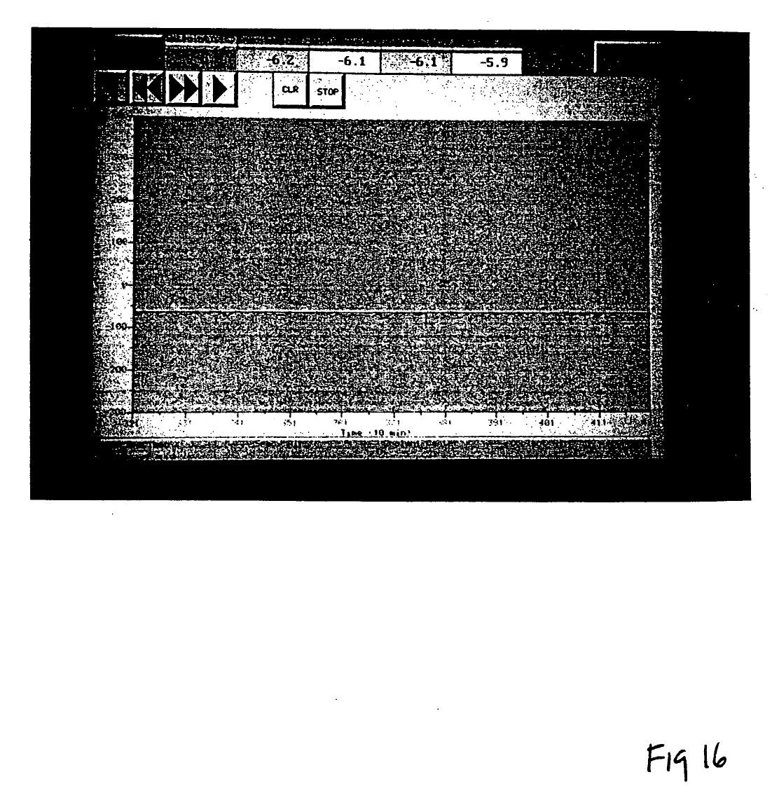

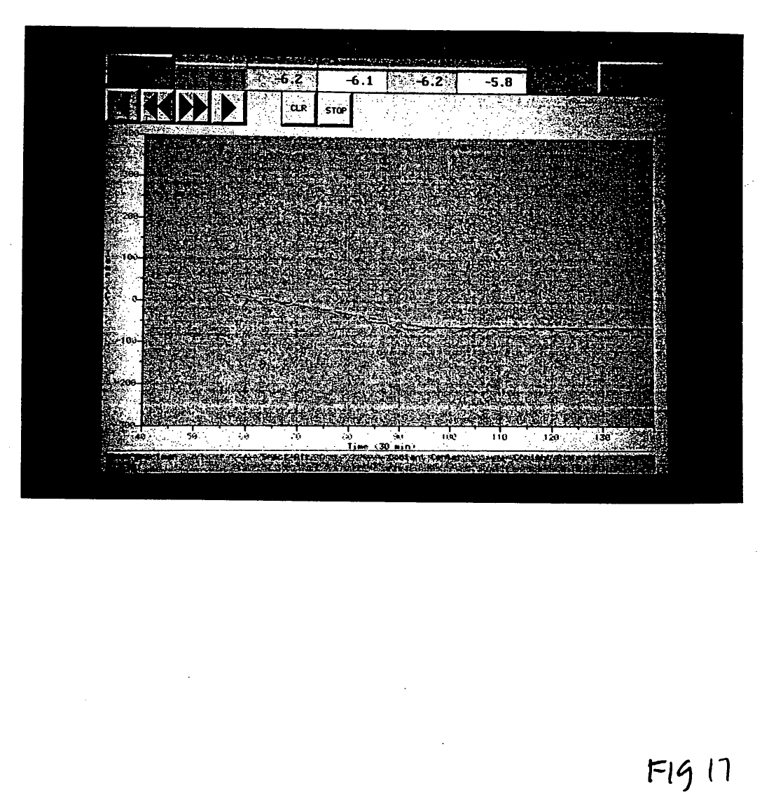

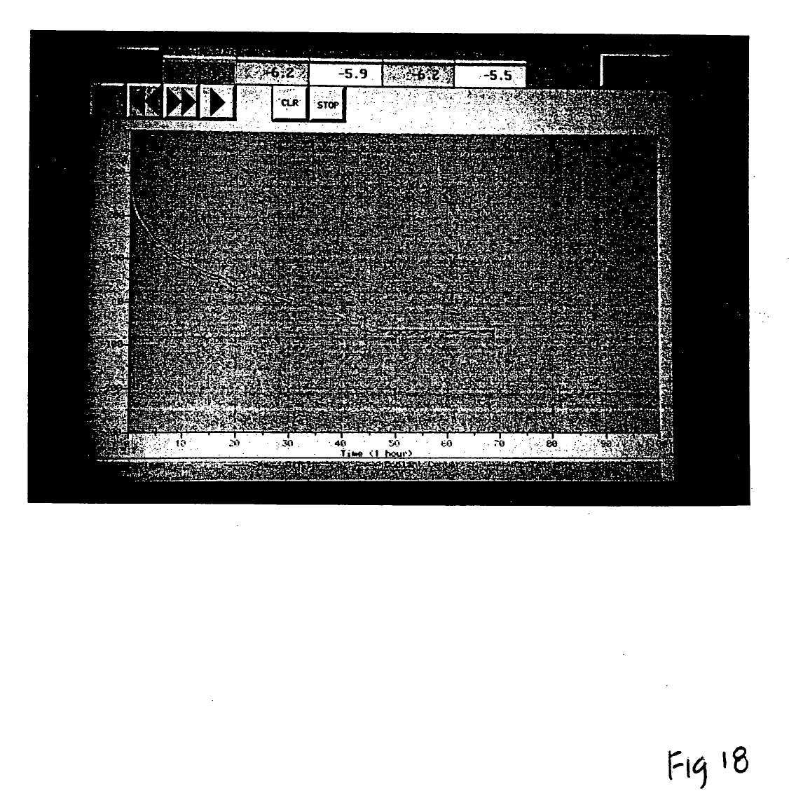

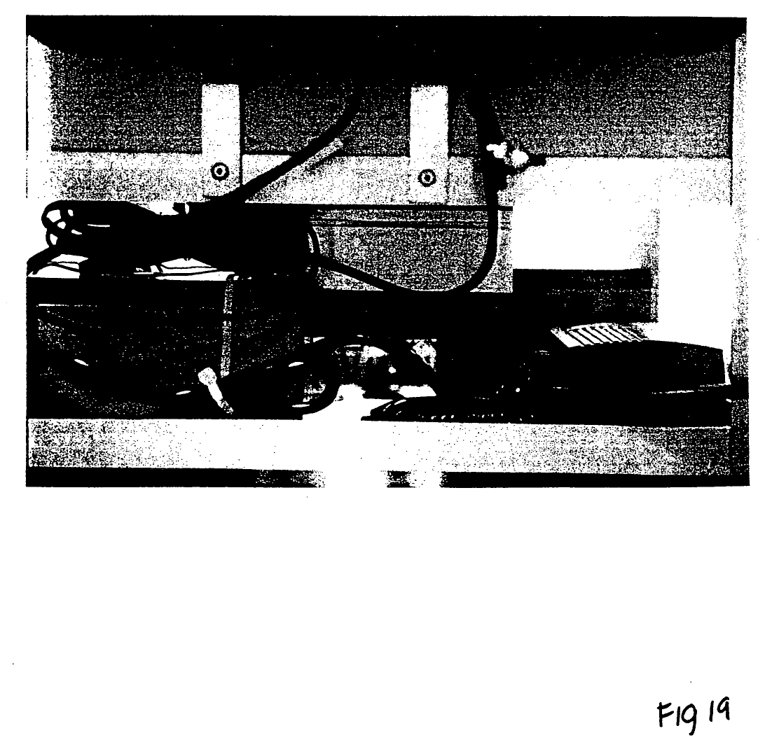

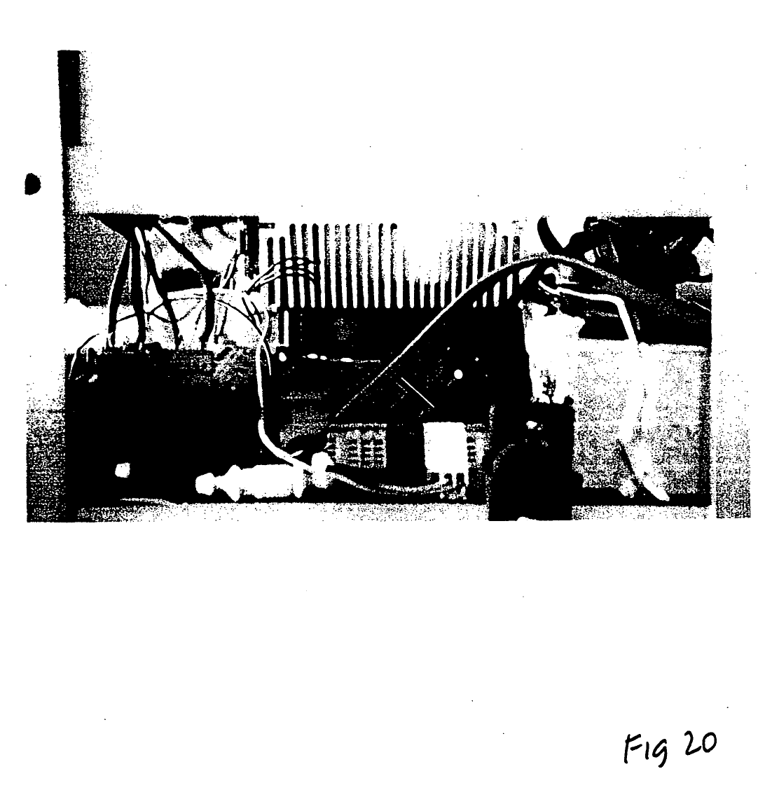

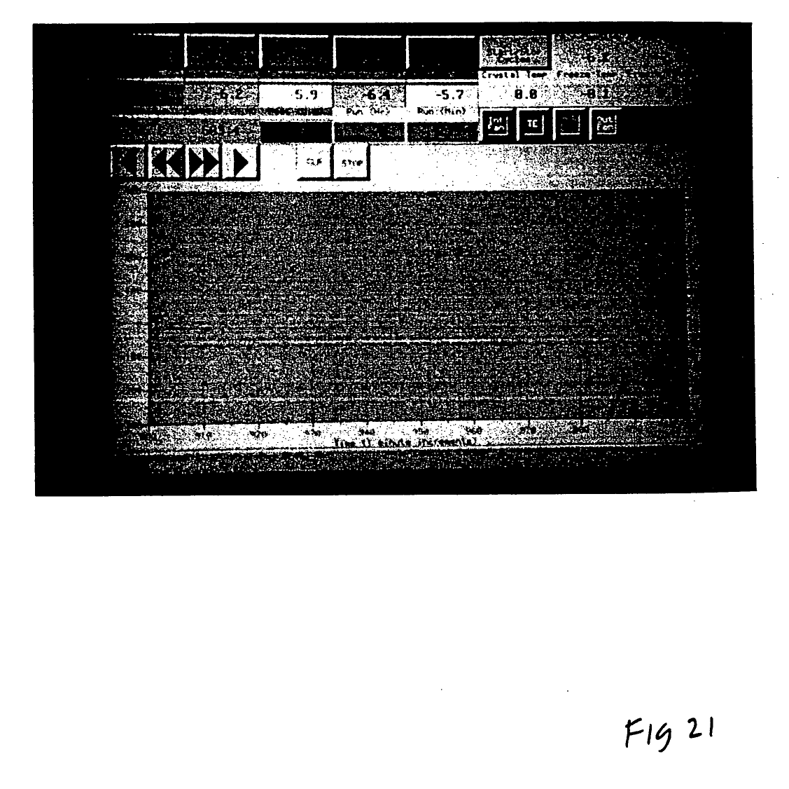

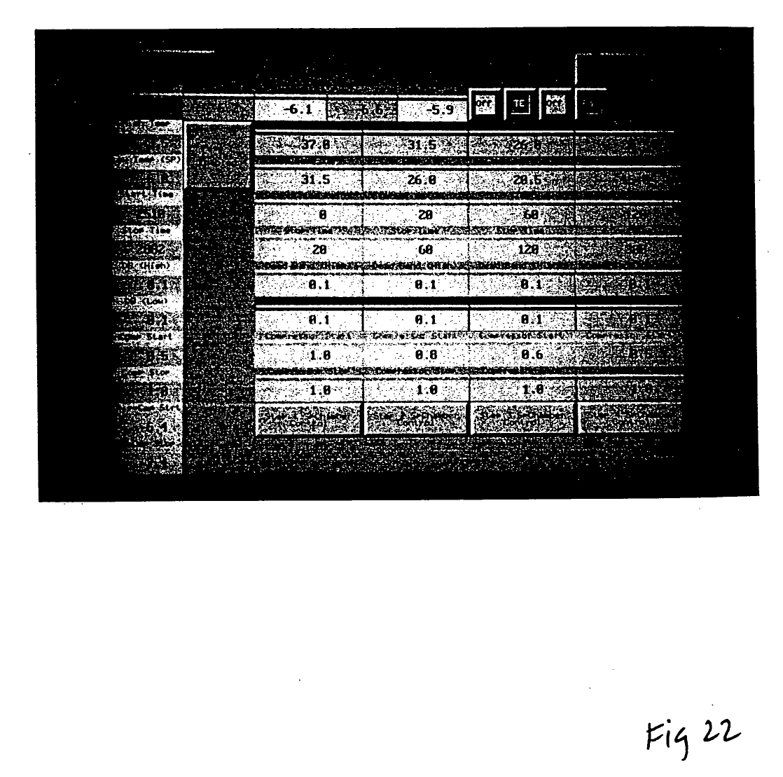

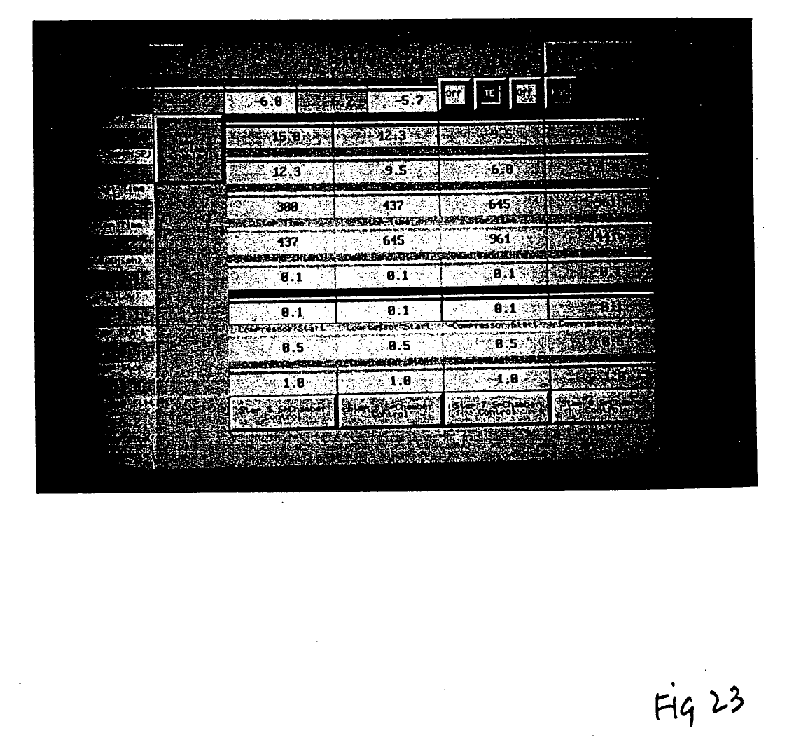

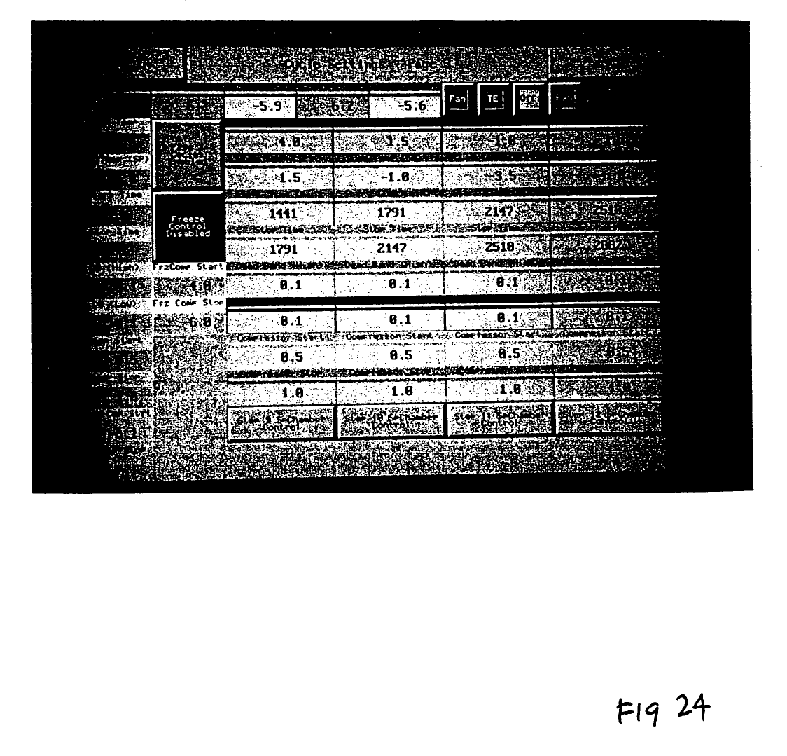

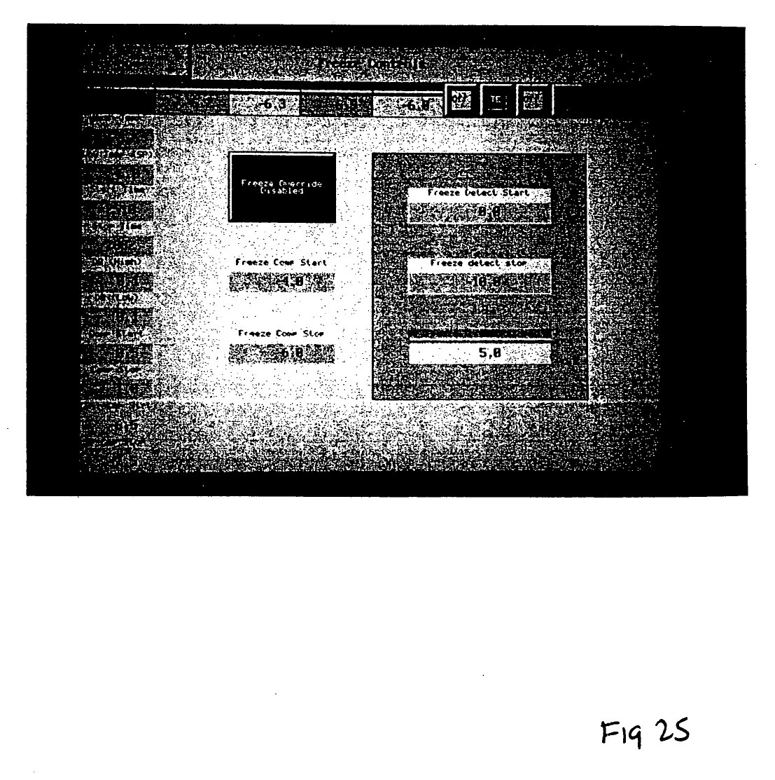

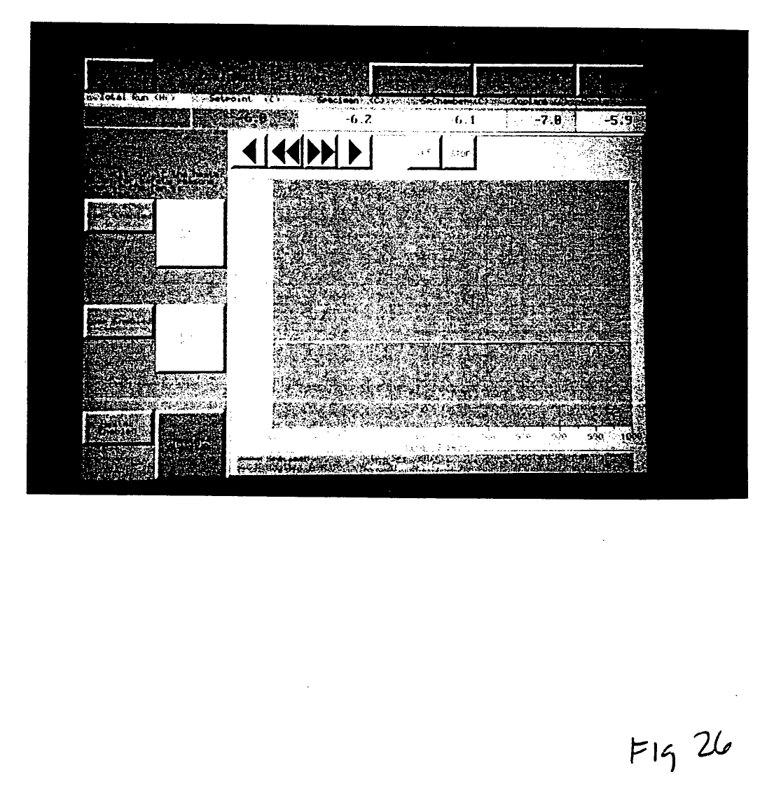

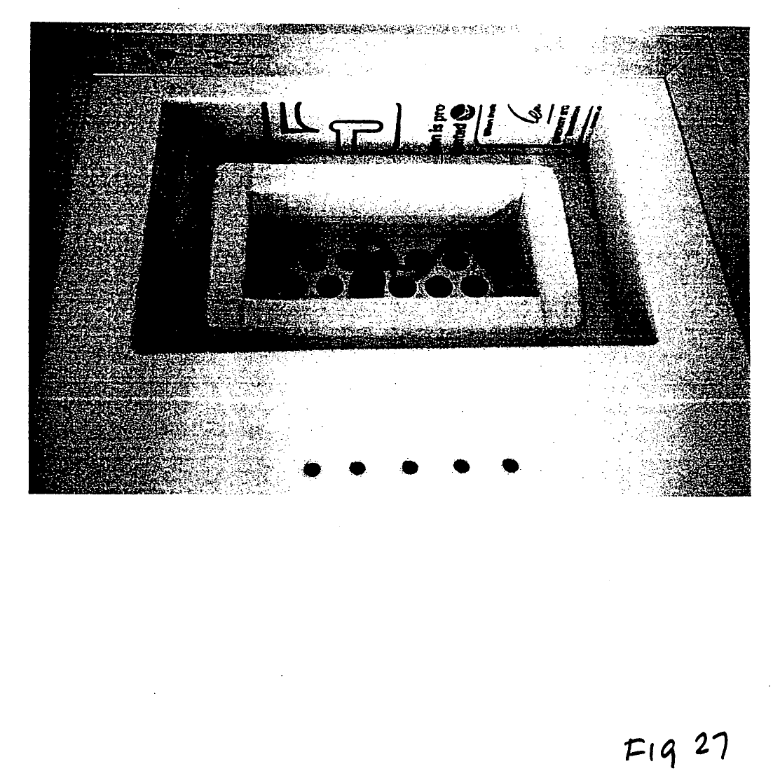

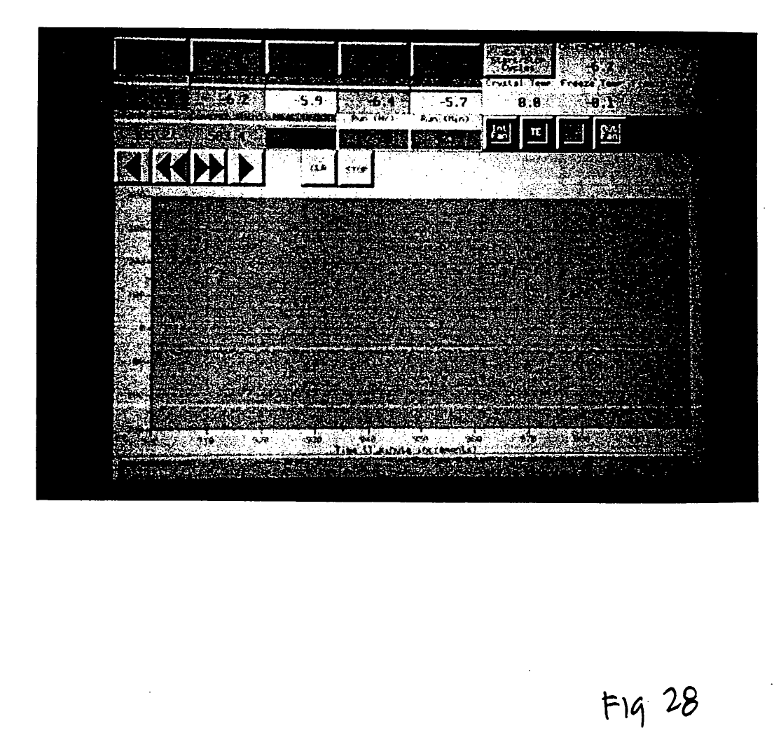

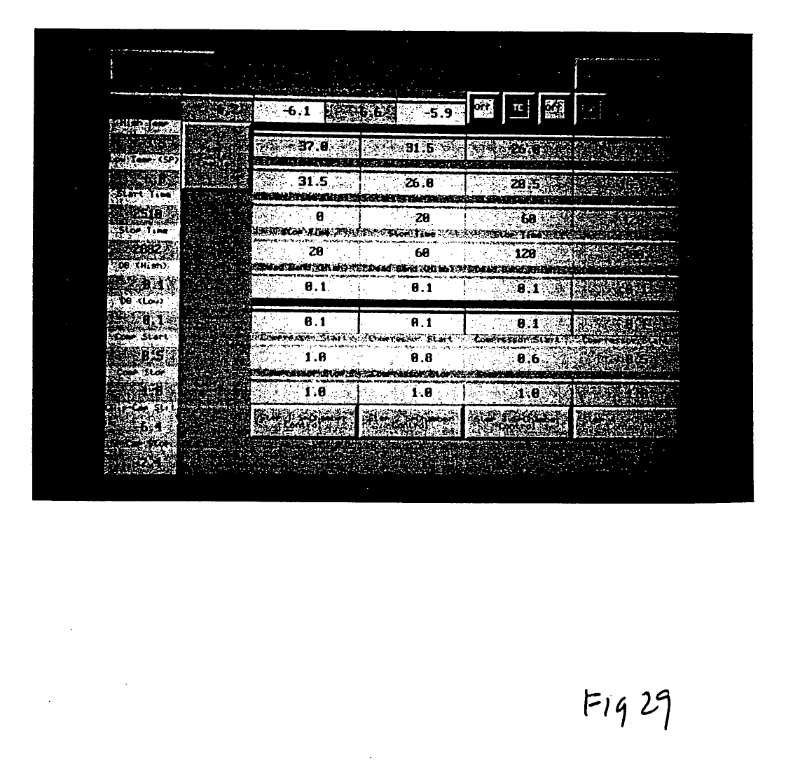

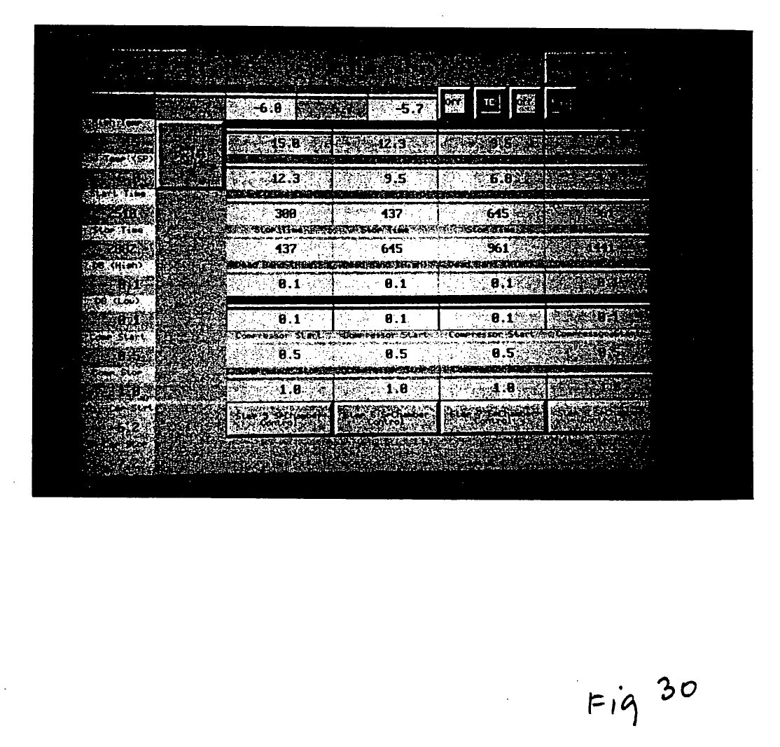

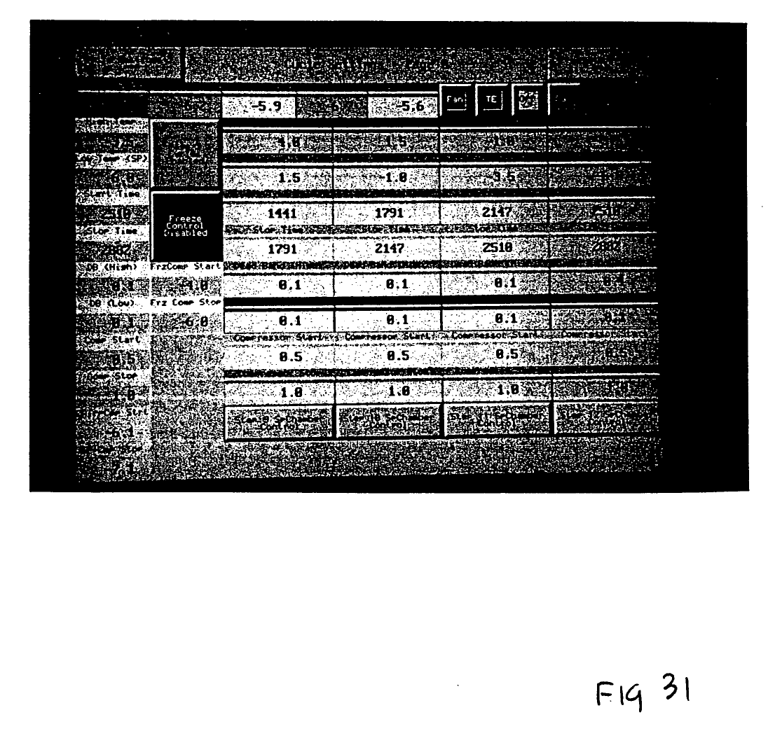

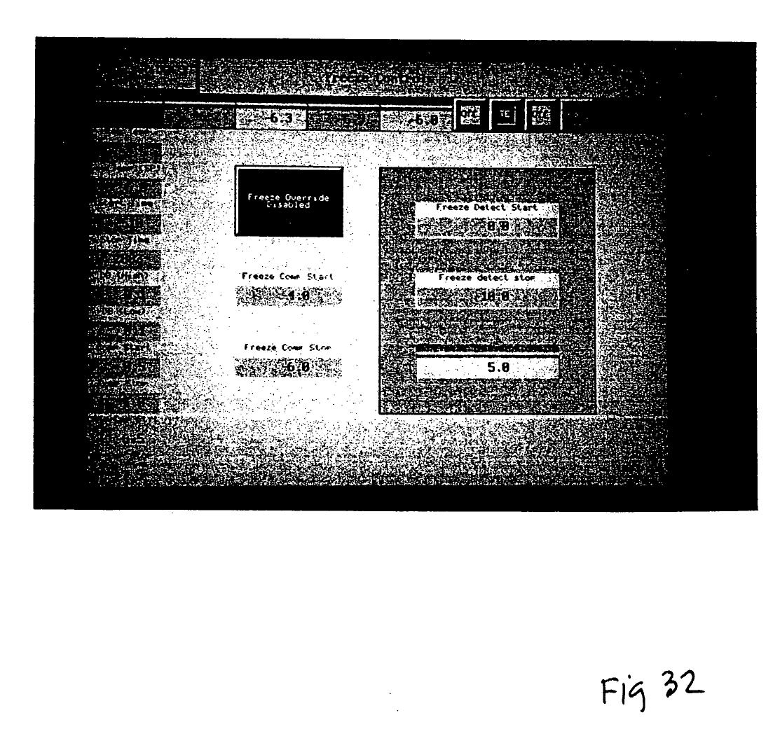

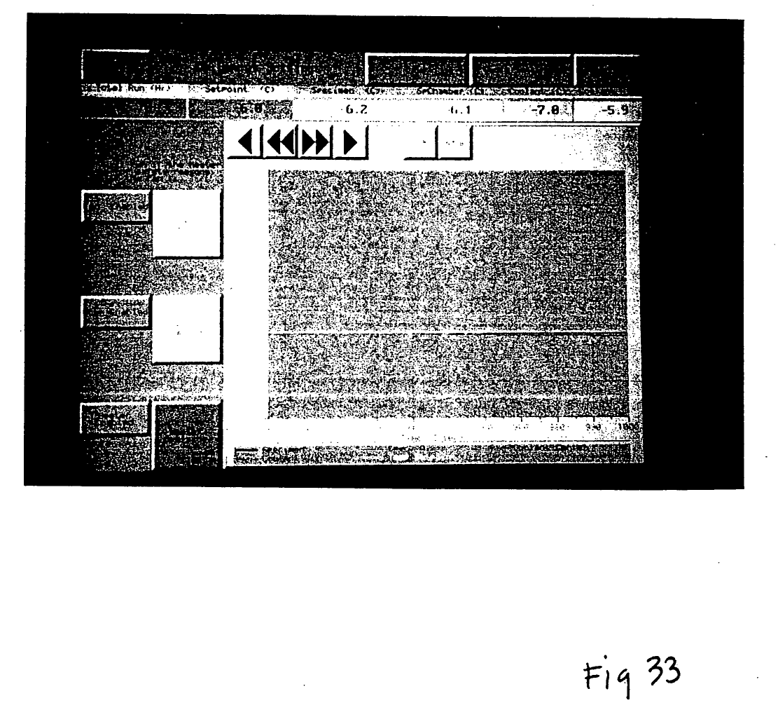

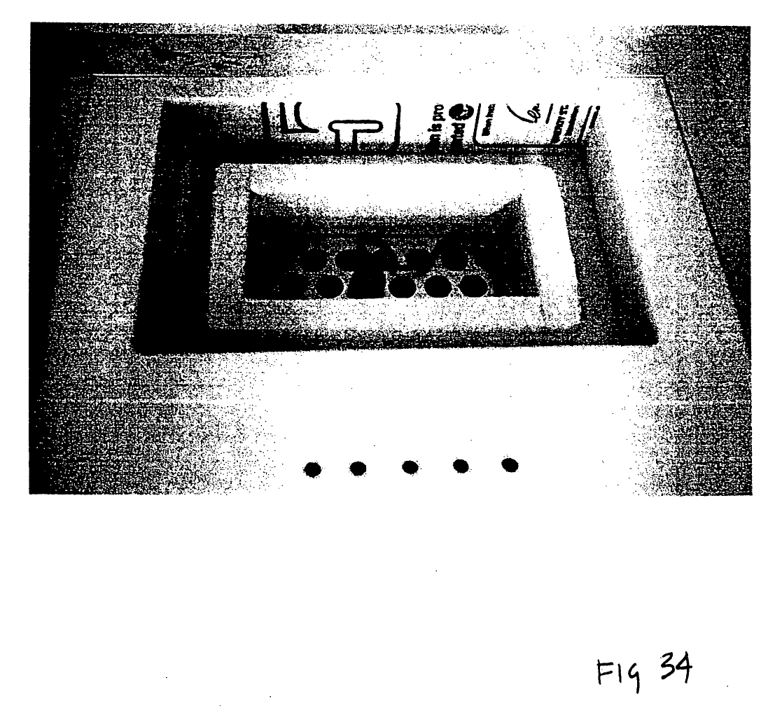

The present invention is showing in diagrammatic form in FIG. 1, and includes a cooling and transportation device 100 having a cooling chamber 102 containing one or more biological samples, such as semen, in a sample holder. These samples are surrounded by coolant air which is intermittently circulated through the cooling chamber across a thermoelectric unit that can cool or heat the circulating air. A fan is used to circulate air through the cooling chamber 102, and a separate fan may be used to introduce and circulate ambient air over the thermoelectric unit in incoming air chamber 104. A sample rotator motor may be used to rotate the samples within the cooling chamber.

A control system includes a PLC controller 200 that receives temperature inputs from the sample holder, ambient air within the cooling chamber, and the thermoelectric unit, as well as possibly within the sample. The PLC receives these temperature inputs, and controls the fans, sample rotator motor, and thermoelectric units such that the temperature of the samples within the cooling chamber are cooled at a predetermined and selectable temperature profile. This profile may be selected through the PLC interface based upon a number of factors, such as the type of sample (equine semen, bovine semen, embryo, cardiac tissues, etc.) the expected period for transport or delay until use, for example.

In a preferred embodiment, a User Interface 106 may be used to select user-selectable preferences for particular applications. For instance, the user may select the type of biological sample being preserved, such as bovine semen. This selection may prompt the PLC to access data from memory 108 which provides specific cooling profile data for a particular application.

The PLC, through the selective control of the fans, motor, and thermoelectric unit, can control the temperature of the biological sample to within a small fraction of a degree, and may adjust the temperature of the sample as a function of time, regardless of outside, or ambient, temperatures.

In use, the biological sample transport and storage device of the present invention may be implemented in several environments, including circumstances wherein the sample must be heated above the ambient temperature as well as circumstances wherein the sample must be cooled below the ambient temperature. Thus the reference to cooling chamber 102 is not to be construed as a limitation on the temperature within the chamber, rather merely indicative of a temperature controlled environmental chamber.

The device of the present invention may reside in a lab and connected to a 120 VAC supply. Alternatively, it can include a portable power source, such as rechargeable batteries, allowing the device to be transported where AC power is not available.

The device of the present invention may be used with any biological material, including but not limited to semen, embryo, ovary, transplant organs. The biological material can be put into this device and cooled and/or heated to follow a programmable temperature/time profile characteristic and most advantageous to the preservation of the specific materials.

The device of the present invention also provides for the use of biological materials stored in suspension fluids, such as extenders for semen and embryos), and due to can be gently agitated allowing maximum beneficial chemical contact. 4) Equipment can manage the physical and chemical environment of any biological specimen therefore with appropriate cycle, extend the useful life of the specimen.

While the particular biological sample transport and storage device as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A biological transport and storage device, comprising:
a cooling chamber containing air and sized to receive one or more biological samples in a sample holder having a temperature sensor;
a thermoelectric unit positioned between the cooling chamber and ambient environment capable of cooling or heating the air within the cooling chamber;
a fan within said cooling chamber and capable of circulating the air through the cooling chamber;
a fan, outside the cooling chamber and adjacent the thermoelectric unit capable of circulating ambient air over the thermoelectric unit;
a temperature sensor within the cooling chamber;
a temperature sensor exposed to ambient temperature; and
a control system includes a PLC controller that receives temperature inputs from the sample holder temperature sensor, the ambient air temperature sensor, and the cooling chamber temperature sensor, and is in electrical communication with the fans and thermoelectric unit such that the temperature of the samples within the cooling chamber are cooled at a predetermined and selectable temperature profile.

2. The device of claim 1, further comprising a sample rotator motor within said cooling chamber and operable on said sample holder to rotate the samples within the cooling chamber.
